(19) United States
(12) Patent Application Publication
(10) Pub. No.: US 2025/0090593 A1
(43) Pub. Date: Mar. 20, 2025

(54) METHOD FOR PRODUCING HYDROGEL MICROBEADS CAPABLE OF MAGNETIC ACTUATION FOR DELIVERY OF THERAPEUTIC SUBSTANCE, HYDROGEL MICROBEADS PRODUCED THEREBY, AND PHARMACEUTICAL COMPOSITION FOR TREATING MUSCULOSKELETAL DISORDERS COMPRISING SAME

(71) Applicant: BIOT KOREA INC., Gwangju (KR)

(72) Inventors: Yeong Jun CHANG, Seoul (KR); Hyeon Soo KIM, Seoul (KR)

(73) Assignee: BIOT KOREA INC., Gwangju (KR)

(21) Appl. No.: 18/965,372

(22) Filed: Dec. 2, 2024

Related U.S. Application Data
(63) Continuation of application No. PCT/KR2023/006062, filed on May 3, 2023.

(30) Foreign Application Priority Data
Jun. 15, 2022 (KR) 10-2022-0072957

(51) Int. Cl. A61K 35/28; A61K 9/06; A61K 9/16; A61K 9/19; A61K 35/32; A61K 38/18

(52) U.S. Cl. CPC A61K 35/28; A61K 9/06; A61K 9/1652; A61K 9/1694; A61K 9/19; A61K 35/32; A61K 38/1841

(57) ABSTRACT

The present invention relates to: a method for producing hydrogel microbeads capable of magnetic actuation for the delivery of a therapeutic substance; hydrogel microbeads produced thereby; and a pharmaceutical composition for treating musculoskeletal disorders, comprising same. The method includes the steps of: (a) producing microbeads by electrospinning a mixed solution including sodium alginate, a biocompatible polymer, and magnetic particles; (b) curing the microbeads through ionic crosslinking; and (c) freeze-drying the cured microbeads.

### CROSS-REFERENCE TO RELATED APPLICATIONS

This application is a continuation of International Application No. PCT/KR2023/006062 filed on May 3, 2023, which claims priority to Korean Patent Application No. 10-2022-0072957 filed on Jun. 15, 2022, the entire contents of which are herein incorporated by reference.

### TECHNICAL FIELD

The present disclosure relates to a method for producing hydrogel microbeads for the delivery of a therapeutic substance, hydrogel microbeads produced thereby, a pharmaceutical composition comprising magnetic particles capable of magnetic actuation and a hydrogel capable of carrying a therapeutic substance, and more particularly, to a method for producing microbeads to which an external magnetic field is applied, hydrogel microbeads produced thereby, and a pharmaceutical composition for treating musculoskeletal diseases comprising the same.

### BACKGROUND ART

It is known that sprains, fractures, contusions, etc. are the most common causes of damage to the musculoskeletal system.

As such when the musculoskeletal system is damaged by degenerative changes or trauma-induced inflammation, and partial or complete rupture, treatments such as surgical treatment to suture the musculoskeletal system, drug treatment such as steroid injections, and physical therapy are known. Among these treatments, drug therapy is limited to relieving local inflammation and attenuating pro-inflammatory factors to provide relief, and non-steroidal anti-inflammatory drugs such as indomethacin, ketoprofen, and ibuprofen, and steroid drugs are mainly used. These non-steroidal anti-inflammatory drugs are known to have the side effects such as exposure to gastrointestinal diseases when used for a long period of time, and steroid drugs are also known to cause side effects such as osteoporosis, hypertension, and neurological complications when used for a long period of time.

Recently, cell therapeutic agents that can solve the problems of existing drug treatments described above and provide a more fundamental treatment for musculoskeletal disorders occurring in tendons, muscles, cartilage, nerves, and bones, etc. through tissue regeneration, have been in the spotlight.

For example, for the regenerative treatment of articular cartilage damage using stem cells, stem cell transplantation has been performed by surgical methods such as microfracture using s chondrocyte or matrix-based chondrocyte transplantation. However, existing methods of transplanting these cells for cartilage regeneration have problems in performing a procedure by exposing defect area by a surgical method, or accurately positioning a large number of cells in the articular cartilage defect area and maintaining the number of cells during the treatment period.

To solve these problems, a method of inserting magnetic particles into stem cells and controlling and directing them in a desired direction using an X-ray image and an external permanent magnet has been proposed. Korean Patent Publication No. 10-2021-0062378 discloses a functional microscaffold that grafts magnetic particles onto a scaffold to enable magnetic actuation for efficient target treatment of various diseases, and a method for preparing the same.

However, according to the afore-mentioned prior art, while cell therapeutic agents can be utilized as main delivery materials, they are not suitable for carrying therapeutic substances such as drugs or growth factors.

### DISCLOSURE

### Technical Problem

The present disclosure has been made in an effort to solve the problem of the prior art as described above, and an object of the present disclosure is to provide a method for producing hydrogel microbeads that may produce hydrogel microbeads capable of carrying not only cells but also therapeutic substances such as drugs or growth factors and capable of magnetic actuation and may control a shape and a swelling degree of the microbeads and mobility of the microbeads in a magnetic field according to types and contents of respective components constituting the microbeads, hydrogel microbeads produced thereby, and a pharmaceutical composition for treating musculoskeletal diseases comprising the same.

### Technical Solution

In order to achieve the above technical challenges, the present disclosure proposes a method for producing microbeads capable of magnetic actuation by an external magnetic field and capable of carrying a therapeutic substance, the method comprising the steps of: (a) producing microbeads by electrospinning a mixed solution including sodium alginate, a biocompatible polymer, and magnetic particles; (b) curing the microbeads through ionic crosslinking; and (c) freeze-drying the cured microbeads, based on a vibration-based special electrospinning technology and an ion curing technology.

In the producing of the microbeads using the electrospinning process performed in the step (a), it is preferable to perform electrospinning at a frequency of 2,000 Hz to 2,400 Hz and a temperature of 30° C. to 80° C. using a cone type nozzle having an inner diameter of 50 μm to 200 μm in order to produce microbeads that can pass through a commonly used syringe needle.

Here, the biocompatible polymer included in the mixed solution provided in the electrospinning process may be one or more selected from the group consisting of, but not necessarily limited to, hyaluronic acid, chitosan, and polyester-based biocompatible polymers.

In addition, the shape, swelling degree, and mobility properties of the microbeads produced may be controlled according to the mixing ratio of sodium alginate and the biocompatible polymer in the mixed solution provided in the electrospinning process. For example, it is preferable that the mixed solution includes 4 to 100 parts by weight of a biocompatible polymer based on 100 parts by weight of sodium alginate.

Further, in the curing of the microbeads through the ionic crosslinking reaction performed in the step (b), the microbeads produced in the step (a) are immersed in a solution containing divalent metal ions (Ca²⁺, Mg²⁺, Sr²⁺, Ba²⁺, etc.) to induce a crosslinking reaction between the anionic functional group of the biocompatible polymer and the divalent metal ions, thereby curing the microbeads.

For example, in this step (b), the microbeads are immersed in a 1 w/v % to 15 w/v % calcium salt solution for 30 minutes to 48 hours to obtain cured microbeads through ionic crosslinking. If the concentration of the calcium salt solution used and the treatment time are lower than the given conditions, insufficient time is allowed for ionic cross-linking and thus intact microbeads are not formed. On the other hand, if the conditions are higher than the given conditions, more ionic crosslinking will occur than necessary, resulting in the microbeads clumping together or deforming.

The method of freeze-drying the hydrogel microbeads in the step (c) is not particularly limited, and a known freeze-drying method may be applied without any limitation.

In another aspect of the present disclosure, the present disclosure proposes hydrogel microbeads produced by the methods.

The hydrogel microbeads according to the present disclosure preferably contain 30 wt % to 70 wt % of magnetic particles to ensure smooth mobility of beads inserted into the body by the application of an external magnetic field. In particular, if the content of magnetic particles exceeds 70 wt %, the microbeads lose their shape during the production step, so that intact microbeads cannot be inserted into the body.

Further, the hydrogel microbeads according to the present disclosure preferably have a swelling degree of 400% to 1,800% when produced under a mixing ratio in the given mixed solution, magnetic particle content, and treatment conditions of a calcium salt solution.

In addition, the hydrogel microbeads according to the present disclosure may have a magnetic strength of 15 emu/g to 40 emu/g and have mobility by application of an external magnetic field when produced under a mixing ratio in the given mixed solution, magnetic particle content, and treatment conditions of a calcium salt solution.

In another aspect of the present disclosure, the present disclosure proposes a pharmaceutical composition for treating musculoskeletal diseases, comprising the hydrogel microbeads and a therapeutic substance carried therein.

The musculoskeletal diseases include cartilage diseases such as osteoarthritis, degenerative arthritis, chondromalacia, and deforming arthrosis arthritis, and rotator cuff tendon diseases such as rotator cuff tear.

In addition, the therapeutic substance carried in the hydrogel microbeads may include, for example, but are not necessarily limited to, autologous bone marrow aspirate concentrates (BMACs), autologous chondrocytes, autologous stem cells (bone marrow stem cells, adipose-derived stem cells), and pain and inflammation-relieving factor (TGF-beta), etc., capable of treating degenerative diseases.

Meanwhile, the pharmaceutical composition for treating musculoskeletal diseases may be an injectable formulation.

When the pharmaceutical composition according to the present disclosure is composed of an injectable formulation, it may include a sterilized aqueous solution, a non-aqueous solvent, a dispersant, a suspending agent, an emulsion, a lyophilized preparation, etc., wherein the non-aqueous solvent and suspending agent may include propylene glycol, polyethylene glycol, vegetable oils such as olive oil, and injectable esters such as ethyl oleate, etc., but are not necessarily limited to the pharmaceutically acceptable carriers described above.

### Advantageous Effects

According to the present disclosure, it is possible to produce hydrogel microbeads capable of carrying not only cells but also therapeutic substances such as drugs or growth factors and capable of magnetic actuation, and furthermore, it is possible to control a shape and a swelling degree of the microbeads and mobility of the microbeads in a magnetic field according to types and contents of respective components constituting the microbeads.

A pharmaceutical composition comprising hydrogel microbeads for the delivery of a therapeutic substance produced in accordance to the present disclosure and an active ingredient comprising a therapeutic substance carried therein is an injectable formulation, etc. and exhibits excellent therapeutic properties for musculoskeletal diseases such as cartilage disease or rotator cuff tendon disease.

### DESCRIPTION OF DRAWINGS

FIGS. 1 to 3 are results showing changes in the shape of microbeads according to the production conditions in the present embodiment, wherein FIG. 1 shows changes in the shape of microbeads according to a mixing ratio of sodium alginate and biocompatible polymer (hyaluronic acid), FIG. 2 shows changes in the shape of microbeads according to an ion curing treatment time, and FIG. 3 shows changes in the shape of microbeads according to an ion curing treatment concentration.

FIG. 4 is results showing actuation performance of microbeads according to the magnetic strength.

### BEST MODE

In describing the present disclosure, when it is decided that a specific description of a related known function and configuration may unnecessarily obscure the gist of the present disclosure, the detailed description will be omitted.

Since embodiments according to the concept of the present disclosure may be variously modified and may have several forms, specific embodiments will be illustrated in the accompanying drawings and will be described in detail in the present specification or application. However, it is to be understood that the present disclosure is not intended to limit embodiments according to the concept of the present disclosure to specific form of disclosure, but includes all modifications, equivalents, and substitutions falling in the spirit and technical scope of the present disclosure.

The terms used in the present specification are used only to describe specific embodiments and are not intended to limited the present disclosure. Singular forms include plural forms unless the context clearly indicates otherwise. It is to be understood that the term “comprises” or “have” as used herein specify the presence of stated features, numerals, steps, operations, components, parts, or combinations thereof, but does not preclude the possibility of the presence or addition of one or more other features, numerals, steps, operations, components, parts, or combinations thereof in advance.

Hereinafter, the present disclosure will be described in more detail with reference to Examples.

Examples according to the present specification may be modified into various different forms, and it is not to be construed that the scope of the present specification is limited to Examples to be described in detail below. Examples according to the present disclosure are provided to more completely describe the present disclosure to one of ordinary skill in the art.

### Examples

### Production of Hydrogel Microbeads

Hydrogel microbeads were produced by implementing hyaluronic acid/sodium alginate/magnetic particle beads using an electrospinning method, and then producing beads with a certain shape through purification and particle size separation. The content of magnetic particles in microbeads plays the biggest role as a parameter in product functionality, such as a shape and a size of the beads, the carrying rate of therapeutic substances, and actuation performance of the beads. Specifically, hyaluronic acid and sodium alginate solutions were prepared separately, and then mixed with magnetic particles according to a mixing ratio. A mixed solution including hyaluronic acid, sodium alginate, and magnetic particles composed of iron oxide was placed in an electrospinning equipment to produce beads under the set spinning conditions (frequency (Hz), voltage (V), and temperature (° C.)) (Table 1). The beads spun through the nozzle were recovered when cured in a 100 mM calcium chloride solution through a collector at the bottom, and then purified to remove impurities and isolate microbeads in the range of 50 to 200 μm by particle size separation.

TABLE 1
Operating conditions of electrospinning equipment for
producing microbeads

| | |
|---|---|
| Frequency (Hz) | 2,200 ± 200 Hz |
| Voltage (V) | 1,200 ± 200 V |
| Temperature (° C.) | 60 ± 2° C. |
| Nozzle type | cone type |
| Nozzle size (μm) | 150 μm |
| Running time (min) | 10 min |

### Shape Control of Microbeads

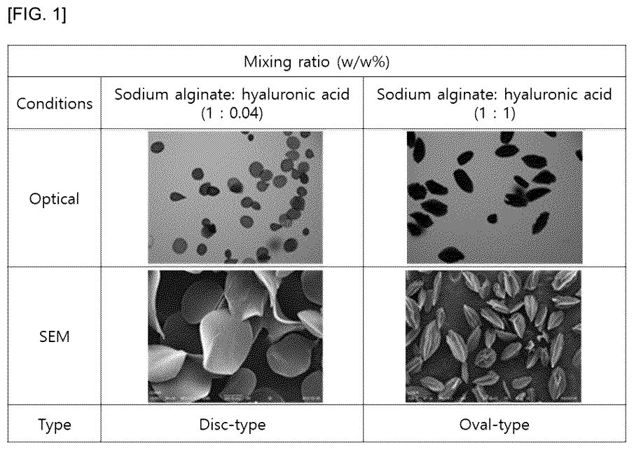

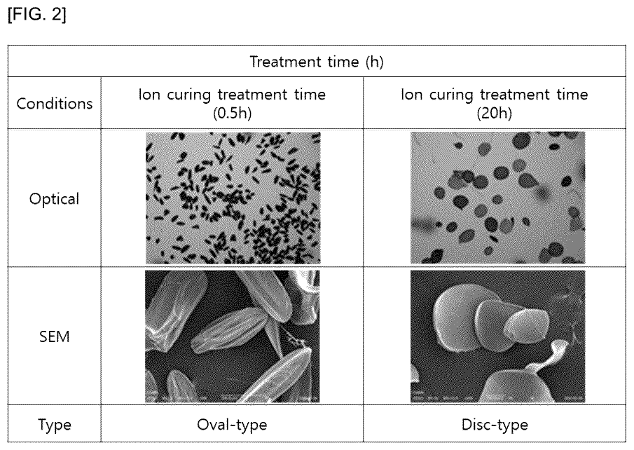

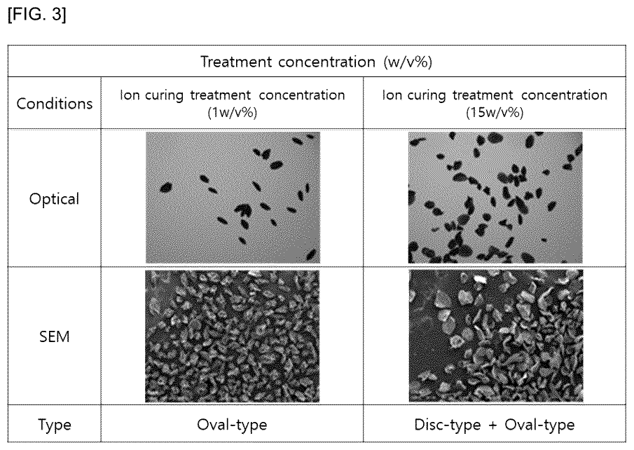

The shape of the microbeads formed during electrospinning was controlled by a mixing ratio between the sodium alginate solution and the hyaluronic acid solution and the ion curing conditions. In order to confirm a shape and a size of the produced beads as a characteristic evaluation for shape control, two analyses were performed. In order to confirm a shape and a size, images of produced and freeze-dried were taken and analyzed using an optical microscope and SEM analysis equipment (FIGS. 1 to 3). Depending on the mixing ratio, it was confirmed that the shape changed from a disk-type to an oval-type as the mixing ratio of the polymer with high moisture absorption rate compared to sodium alginate increased (FIG. 1). It was confirmed that the shape changed from an oval-type to a disk-type as the treatment time increased during ion curing (FIG. 2). In addition, for the treatment concentration during ion curing, it was confirmed that a production rate of a disk-type increased as the concentration increased regardless of the treatment time (FIG. 3).

### Evaluation of Moisture Swelling Degree for Microbeads

The swelling degree of microbeads formed after electrospinning was controlled according to the ion curing conditions. In order to confirm the swelling degree as an evaluation method for the control of swelling properties, a swelling degree analysis according to moisture absorption was performed. It was confirmed that the swelling degree decreased while the moisture absorbency decreased as the treatment time increased during ion curing (Table 2). On the other hand, for the treatment concentration during ion curing, it was confirmed that the swelling degree increased while the moisture absorbency improved while as the concentration increased (Table 3).

TABLE 2
Changes in swelling degree of microbeads according
to ion curing treatment time
Treatment time (h)

| | Ion curing | Ion curing |
|---|---|---|
| | treatment time | treatment time |
| Conditions | (0.5 h) | (20 h) |
| Swelling rate | 1,251% | 613% |

TABLE 3
Changes in swelling degree of microbeads according
to ion curing treatment concentration
Treatment concentration (w/v %)

| | Ion curing | Ion curing |
|---|---|---|
| | treatment concentration | treatment concentration |
| Conditions | (1 w/v %) | (15 w/v %) |
| Swelling rate | 1,254% | 1,889% |

### Evaluation of Actuation Performance for Microbeads

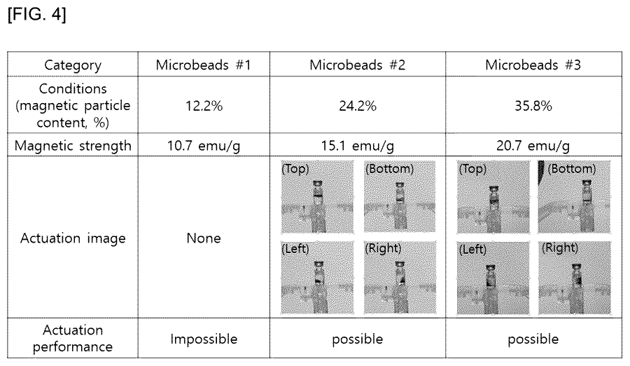

The actuation performance of microbeads was controlled according to the magnetic strength. In order to evaluate the actuation performance, the magnetic strength was confirmed using VSM analysis equipment and the actuation in an external magnetic field was evaluated. If the external magnetic field strength is 300 Gauss, three different types of microbeads with different magnetic strengths were produced to evaluate whether they were actuated (FIG. 4). The actuation performance was confirmed through images whether it was actuated according to four directions (top, bottom, left, and right). It was confirmed that actuation was possible when the magnetic strength was at least 15 emu/g or more (based on a field value of 1 k Oe).

The present disclosure is not limited to the above embodiments, but can be prepared in various different forms. A person having ordinary skill in the art to which the present disclosure pertains will understand that the present disclosure can be implemented in other specific forms without changing the technical spirit or essential features of the present disclosure. Therefore, it is to be understood that the embodiments described above are illustrative rather than being restrictive in all aspects.

### INDUSTRIAL APPLICABILITY

According to the present disclosure, it is possible to produce hydrogel microbeads capable of carrying not only cells but also therapeutic substances such as drugs or growth factors and capable of magnetic actuation, and furthermore, it is possible to control a shape and a swelling degree of the microbeads and mobility of the microbeads in a magnetic field according to types and contents of respective components constituting the microbeads.

What is claimed is:

1. A method for producing hydrogel microbeads, comprising the steps of:
(a) producing microbeads by electrospinning a mixed solution including sodium alginate, a biocompatible polymer, and magnetic particles;
(b) curing the microbeads through ionic crosslinking; and
(c) freeze-drying the cured microbeads.

2. The method of claim 1, wherein in the step (a), electrospinning is performed at a frequency of 2,000 Hz to 2,400 Hz and a temperature of 30° C. to 80° C. using a cone type nozzle having an inner diameter of 50 μm to 200 μm.

3. The method of claim 1, wherein the biocompatible polymer is one or more selected from the group consisting of hyaluronic acid, chitosan, and polyester-based biocompatible polymers.

4. The method of claim 1, wherein the mixed solution includes 4 to 100 parts by weight of the biocompatible polymer based on 100 parts by weight of sodium alginate.

5. The method of claim 1, wherein in the step (b), the microbeads are cured through ionic crosslinking in a 1 w/v % to 15 w/v % calcium salt solution for 30 minutes to 48 hours.

6. A hydrogel microbead produced by the method of claim 1.

7. The hydrogel microbead of claim 6, wherein the hydrogel microbead contains 30 wt % to 70 wt % of magnetic particles.

8. The hydrogel microbead of claim 6, wherein the hydrogel microbead has a swelling degree of 400% to 1,800%.

9. The hydrogel microbead of claim 6, wherein the hydrogel microbead has a magnetic strength of 15 emu/g to 40 emu/g and have mobility by application of a magnetic field.

10. A pharmaceutical composition for treating cartilage disease or rotator cuff tendon disease, comprising the hydrogel microbead of claim 6 and a therapeutic substance carried in the hydrogel microbead.

11. The pharmaceutical composition of claim 10, wherein the cartilage disease is any one selected from the group consisting of osteoarthritis, degenerative arthritis, chondromalacia, and deforming arthrosis arthritis.

12. The pharmaceutical composition of claim 10, wherein the therapeutic substance is autologous bone marrow aspirate concentrates (BMACs), autologous chondrocytes, autologous stem cells (bone marrow stem cells, adipose-derived stem cells), and pain and inflammation-relieving factor (TGF-beta), capable of treating degenerative diseases.

13. The pharmaceutical composition of claim 10, wherein the pharmaceutical composition is an injectable formulation.
